(19) United States
(12) Patent Application Publication
(10) Pub. No.: US 2009/0254081 A1
(43) Pub. Date: Oct. 8, 2009

(54) SYSTEM AND METHOD FOR SURGICAL JAW ASSEMBLY

(75) Inventors: Jeffrey Wayne ALLISON, Etters, PA (US); Keith Edwin MILLER, Manheim, PA (US)

Correspondence Address: TYCO TECHNOLOGY RESOURCES, 4550 NEW LINDEN HILL ROAD, SUITE 140, WILMINGTON, DE, 19808-2952, US

(73) Assignee: TYCO ELECTRONICS CORPORATION, Berwyn, PA (US)

(21) Appl. No.: 12/099,226

(22) Filed: Apr. 8, 2008

(51) Int. Cl. A61B 19/00; B23P 21/00

(52) U.S. Cl. 606/39; 29/469

(57) ABSTRACT

A surgical jaw assembly having a top portion and a bottom portion hingedly attached. The top and bottom portion having a base, a seal plate and a cover, the seal plate and base being in direct contact and being electrically common. The bottom portion having a blade that is stationary relative to the base and the top portion having an insert configured to receive the blade when the top portion and bottom portion are in a predetermined position. The cover is overmolded onto the base, securing the seal plate to the base on both the top portion and bottom portion.

### FIELD OF THE INVENTION

The present invention is directed to a surgical jaw assembly. More specifically, the present invention is directed to a surgical jaw assembly with an overmold.

### BACKGROUND OF THE INVENTION

Electrosurgical forceps use both mechanical clamping action and electrical energy to affect hemostasis by heating tissue and blood vessels to coagulate, cauterize and/or seal tissue. Instruments, such as a surgical jaw, are inserted into the patient's body to facilitate various tasks during surgical procedures, such as cutting or ligating blood vessels or vascular tissue. Due to the inherent spatial considerations of the surgical cavity, surgeons often have difficulty suturing vessels or performing other traditional methods of controlling bleeding, e.g., clamping and/or tying-off transected blood vessels. By using a surgical jaw assembly, a surgeon can cauterize, coagulate/desiccate and/or reduce or slow bleeding simply by controlling the intensity, frequency and duration of the electrosurgical energy applied through the jaw members to the tissue.

For the purposes herein, “coagulation” is defined as generally a process of desiccating tissue in which the tissue cells are ruptured and dried. “Vessel sealing” or “tissue sealing” is defined generally as the process of liquefying collagen in the tissue so that it reforms into a fused mass. Coagulation of small vessels is ordinarily sufficient to permanently close them, while larger vessels typically need to be sealed to assure permanent closure.

In order to effectively seal vessels (or tissue) two predominant mechanical parameters must be accurately controlled—the pressure applied to the vessel (tissue) and the gap distance between the electrodes—both of which are affected by the thickness of the sealed vessel. More particularly, accurate application of pressure is important to oppose the walls of the vessel; to reduce the tissue impedance to a low enough value that allows enough electrosurgical energy through the tissue; to overcome the forces of expansion during tissue heating; and to contribute to the end tissue thickness which is an indication of a good seal.

Some embodiments of surgical jaw assemblies used for surgical procedures require adhesives, glues or other fasteners to secure the various components of the surgical jaw assembly. During manufacture of the assemblies, each assembly may receive varying amounts of adhesive, resulting in varying thicknesses of adhesive between each electrically conductive surface. A custom stop surface must be added to each assembly after manufacture to provide an accurate predetermined gap distance between the electrically conductive surfaces. The addition of the custom stop surface is inefficient and expensive. Other embodiments of surgical jaw assemblies include forming a premolded datum on a base before assembling the remaining components. The premolded datum adds extra time and costs to manufacturing, however. Therefore what is needed is a surgical jaw assembly without adhesives, glues or fasteners to secure the various components in the surgical jaw assembly. What is also needed is a surgical jaw assembly with a stop surface that is integral with the components.

### SUMMARY OF THE INVENTION

The present invention is directed to a surgical jaw assembly having a bottom portion. The bottom portion has a first base, a blade disposed in the first base, and a first seal plate disposed on the first base. The first seal plate has an aperture and a flanged edge. The bottom portion also has a first cover at least partially covering the first base. The surgical jaw assembly also has a top portion that has a second base, an insert disposed in the second base, and a second seal plate disposed on the second base. The second seal plate has an aperture and a flanged edge. A second cover at least partially covers the second base. The first seal plate is in contact with the first base and the second seal plate is in contact with the second base. The first cover and second cover covers at least a portion of the first base and at least a portion of the second base, and the first cover secures the blade and first seal plate to the first base by engaging the flanged edge of the seal plate. The second cover secures the insert and second seal plate to the second base by engaging the flanged edge of the seal plate.

The present invention is also directed to a method of manufacturing a surgical jaw assembly having the steps of providing a bottom portion having a first base, a blade, a first seal plate and a first cover. The blade is disposed in the first base and the first seal plate is disposed on the first base such that the first seal plate contacts the first base. The method also includes the step of providing a top portion with a second base, an insert, a second seal plate and a second cover. The insert is disposed in the second base and the second seal plate is disposed on the second base such that the second seal plate contacts the second base. The method also includes overmolding a first cover at least partially surrounding the first base. The first cover secures the blade and first seal plate to the first base such that the first cover engages the first seal plate. Lastly, the method includes overmolding a second cover at least partially surrounding the second base. The second cover secures the insert and second seal plate to the second base such that the second cover engages the second seal plate.

The present invention is further directed to a method of manufacturing a surgical jaw assembly having the steps of providing at least one base and aligning a seal plate with a flanged edge atop the at least one base, such that the seal plate is in contact with the at least one base. Molding a cover at least partially surrounding the at least one base, where the cover secures the seal plate to the at least one base by engaging with the flanged edge of the seal plate.

An advantage of the present invention is the omission of adhesives or fasteners from the assembly to secure the components to one another, thereby producing a precise surgical jaw assembly that is capable of providing the desired pressure and gap distance for procedures.

Yet another advantage of the present invention is the use of injection molding to manufacture the components, thereby creating more uniformity and precision with multiple surgical jaw assemblies.

Still another advantage of the present invention is the use of a stop surface or other feature that is integrated into the surgical jaw assembly, thereby creating more uniformity and precision in the gap distance of surgical jaw assemblies.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 shows an exemplary embodiment of a surgical jaw assembly.

FIG. 2 shows an exemplary embodiment of a top portion of the surgical jaw assembly of FIG. 1.

FIG. 3 shows a cross sectional view of an exemplary embodiment of a top portion of a surgical jaw assembly.

FIG. 4 shows an exemplary embodiment of a bottom portion of the surgical jaw assembly of FIG. 1.

FIG. 5 shows exemplary embodiments of individual components of the bottom portion of the surgical jaw assembly of FIG. 4.

FIG. 6 shows an exemplary embodiment of a base of a surgical jaw assembly.

FIG. 7 shows a cross-sectional view of an exemplary embodiment of a bottom portion of a surgical jaw assembly.

FIG. 8 shows an exemplary embodiment of a bottom portion of a surgical jaw assembly.

FIG. 9 shows an exemplary embodiment of a bottom portion of a surgical jaw assembly without an overmold.

Wherever possible, the same reference numbers will be used throughout the drawings to refer to the same or like parts.

### DETAILED DESCRIPTION OF THE INVENTION

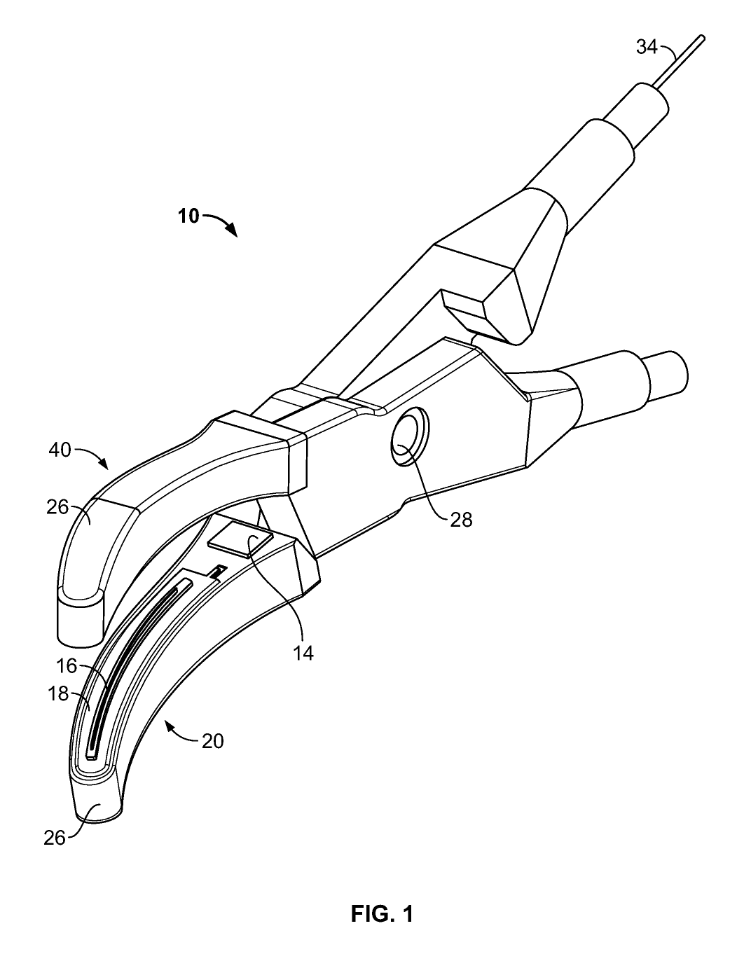

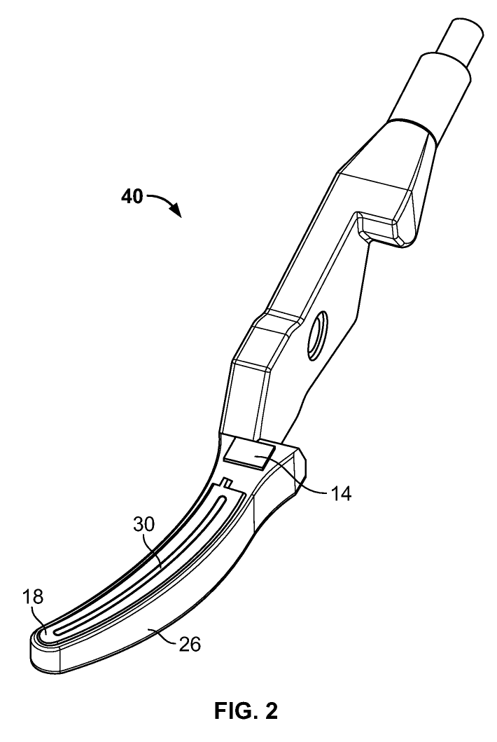

FIG. 1 shows a surgical jaw assembly 10 with a top portion 40 and bottom portion 20. Top portion 40 and bottom portion 20 are hingedly attached to one another and may rotate about a pivot point 28. A fastener or other suitable type of securing device (not shown) may be used to connect and secure top portion 40 to bottom portion 20 and allow movement about pivot point 28. Top portion 40 may extend into pivot point 28 to engage with bottom portion 20, as shown in FIG. 1. Bottom portion 20 includes a blade 16 and seal plate 18, where blade 16 protrudes through an aperture 30 (FIG. 2) in seal plate 18. Blade 16 is stationary relative to bottom portion 20 and may be an electrically charged blade. The movement of top portion 40 and bottom portion 20 about pivot point 28 facilitates the severing or cutting of vessels or tissue (not shown) by blade 16. Seal plate 18 coagulates or cauterizes the tissue or vessel on either side of the cut made by blade 16. Cauterizing by seal plate 18 substantially prevents, reduces or eliminates bleeding in the vessel or tissue (not shown). When top portion 40 and bottom portion 20 are rotated about pivot point 28, blade 16 contacts, or substantially contacts, top portion 40 through the vessel or tissue (not shown). An electrical charge is applied to blade 16 to sever or cut the tissue (not shown). In addition, an electrical charge is applied to seal plate 18 to cauterize the tissue (not shown) on either side of the cut made by blade 16.

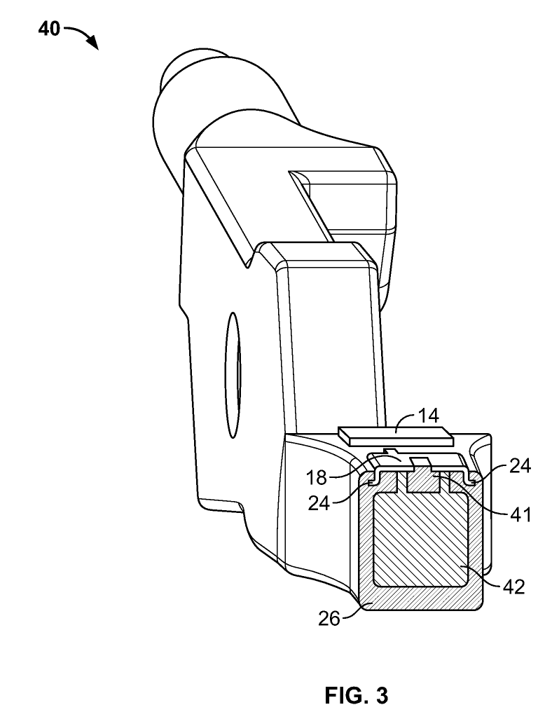

Surgical jaw assembly 10 has a first position and a second position and a plurality of predetermined positions in between. The first position may be an open position (FIG. 1) and the second position may be a closed position (not shown). In the open position, a vessel or tissue may be placed between top portion 40 and bottom portion 20. Seal plates 18 are positioned to create a predetermined gap and coagulate the tissue on either side of the cut made by blade 16 to prevent bleeding. When surgical jaw assembly is in the closed position, blade 16 substantially contacts insert 41 (FIG. 3) through aperture 30 of seal plate 18 on top portion 40 after an electrical charge has been applied to blade 16 and the tissue is cut. Blade 16 may substantially press into insert 41 when surgical jaw assembly 10 is in the closed position.

FIGS. 2 and 3 show top portion 40 with a base 42 (FIG. 3) substantially surrounded by cover 26 for securing components of top portion 40 of surgical jaw assembly 10 together. Cover 26 may also insulate components of top portion 40 and may be plastic or other non-conductive material. Base 42 may be a metal or other suitable conductive material manufactured from an injection mold process, machine process, stamping process or other suitable process. Top portion 40 may also include a seal plate 18 with aperture 30 and an insert 41 for substantially contacting or receiving blade 16 when the top portion 40 and bottom portion 20 are in a predetermined position. Insert 41 is disposed in base 42, and then seal plate 18 is disposed on base 42, over insert 41 such that seal plate 18 is in direct contact with base 42. In addition, seal plate 18 and base 42 are electrically common, thus no separation between seal plate 18 and base 42 is necessary. Cover 26 is then applied to secure seal plate 18 and insert 41 to base 42 without the use of additional adhesives or fasteners. When seal plate 18 is disposed on base 42, insert 41 is visible through aperture 30. Insert 41 may also protrude through aperture 30. Seal plate 18 may also have flanged edges 24 to engage in cover 26 to secure seal plate 18 to base 42. Insert 41 may be a liquid silicone rubber or other suitable material. Cover 26 may be injection molded to top portion 40.

Top portion 40 and bottom portion 20 may include a stop surface 14. On the top portion 40, stop surface 14 may be disposed on base 42 or may be unitary with base 42. On bottom portion 20, stop surface 14 may be disposed on a base 12 (FIG. 5), and may be unitary with base 12. Stop surface 14 may also be unitary with the cover 26 on both top portion 40 and bottom portion 20. Stop surface 14 maintains a predetermined distance between top portion 40 and bottom portion 20 when surgical jaw assembly 10 is in the closed position. Top portion 40 and bottom portion 20 may each include a stop surface 14, or a stop surface 14 may be disposed on either the top or bottom portion only.

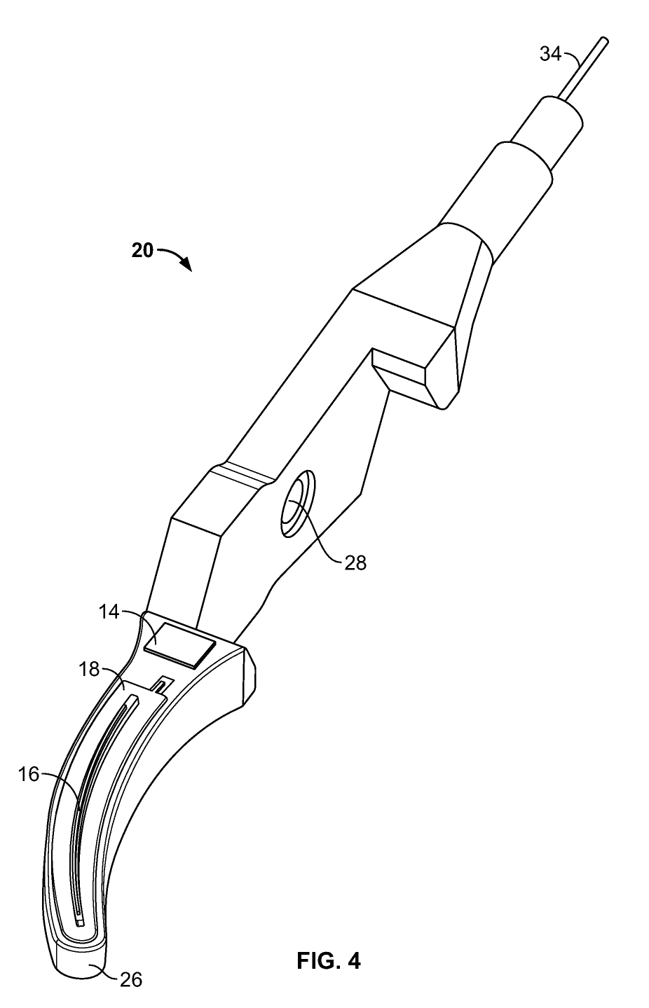

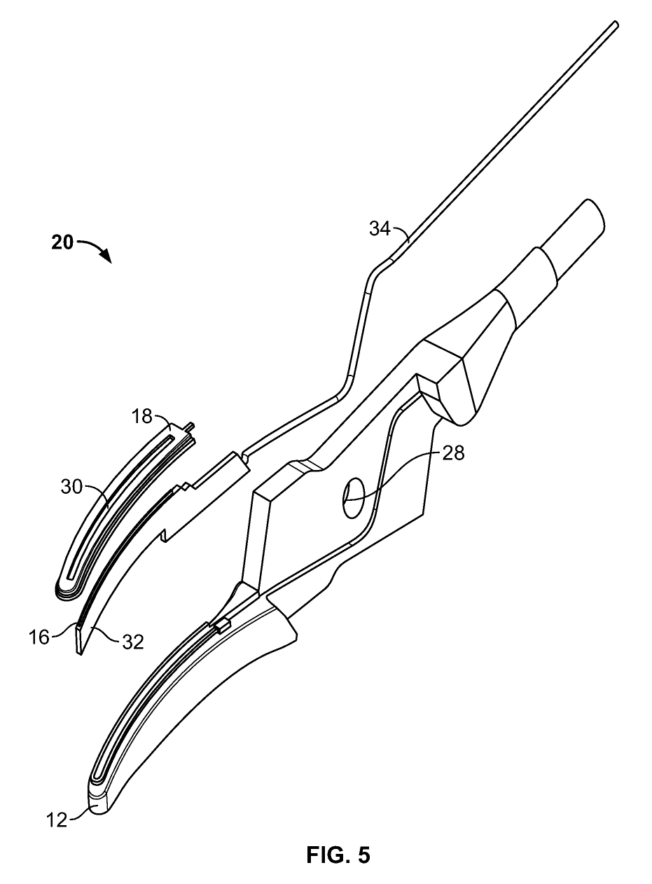

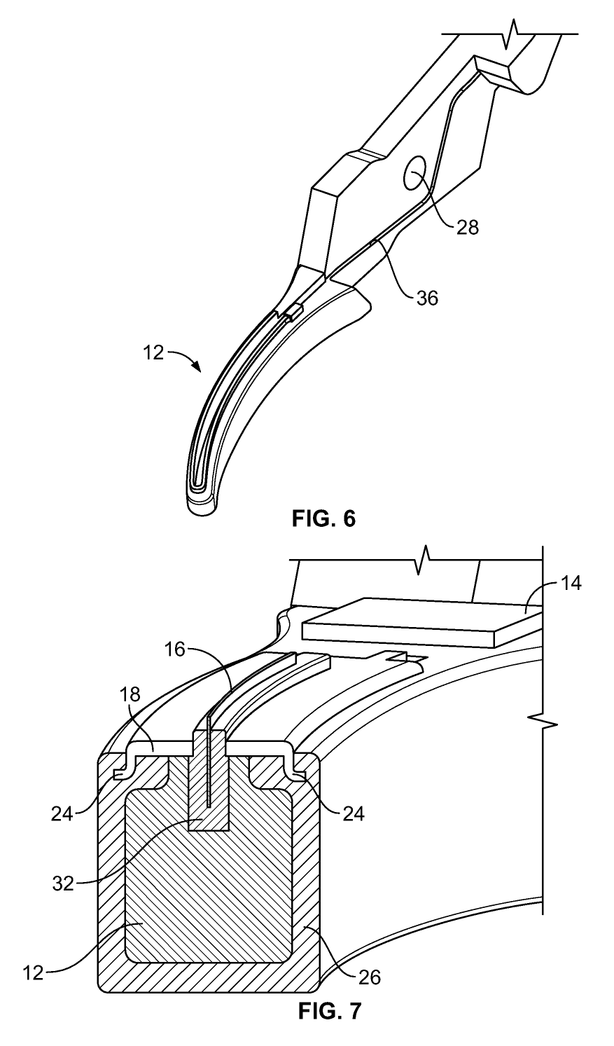

FIGS. 4 through 6 show bottom portion 20 of surgical jaw assembly 10 having a base 12, a blade 16, a seal plate 18 and a cover 26. Base 12 may be a metal or other suitable conductive material manufactured from an injection mold process, machine process, stamping process or other suitable process. Blade 16 is disposed in base 12, with a blade overmold 32 (FIG. 5) substantially surrounding blade 16 and forming a base for blade 16. Blade 16 may be an electrically charged or electrically conductive blade and blade 16 may be overmolded prior to being disposed in base 12. Blade overmold 32 may be manufactured using liquid silicone or any other suitable material. Blade 16 has a conductive wire 34 for conducting electricity to blade 16. Wire 34 is disposed in base 12 in a recessed channel 36 (FIG. 6). Recessed channel 36 retains wire 34 and protects wire 34 from external forces or damage.

Seal plate 18 has an aperture 30 for accepting blade overmold 32 and blade 16. Blade 16 may be used for cutting or severing a vessel or other article. Seal plate 18 is placed over blade 16 and is disposed on base 12. Seal plate 18 is in direct contact with base 12 and electrically common with base 12, thus no separation between seal plate 18, and base 12 is necessary. When seal plate 18 is placed over blade 16, blade 16 is disposed in aperture 30. Blade 16 may securely fit into aperture 30 where there is substantially no additional space between blade 16 and seal plate 18 and such that blade 16 and seal plate 18 are not easily separable from one another once assembled. Alternately, blade 16 and seal plate 18 may be securely assembled such that they are easily removable from each other, when a force is applied to either blade 16 or seal plate 18. Seal plate 18 has flanged edges 24 (FIG. 7) to assist in securing seal plate 18 to base 12.

Bottom portion 20 also includes cover 26. Cover 26 substantially surrounds base 12 and secures seal plate 18 and blade 16 to base 12. No additional adhesives or bonds or fasteners are necessary to secure seal plate 18 and blade 16 to base 12. Cover 26 may be plastic or other suitable material. To provide a secure fit to base 12, and seal plate 18, cover 26 may be injection molded to bottom portion 20. In addition to a secure fit, an injection molding process provides uniformity during manufacturing when a plurality of bottom portions 20 are produced. While cover 26 secures blade 16 to base 12, cover 26 may not directly contact blade 16. Alternatively, cover 26 may directly contact blade 16. Blade overmold 32 is disposed in base 12, seal plate 18 is disposed on top of base 12, with blade 16 protruding through aperture 30 of seal plate 18, and cover 26 substantially directly contacts seal plate 18 and base 12.

FIG. 7 illustrates a cross sectional view of bottom portion 20. As described in detail with respect to FIGS. 4 through 6, bottom portion 20 is configured with blade 16 disposed in base 12 and seal plate 18 resting on base 12. Blade 16 protrudes through aperture 30 (FIG. 5) in seal plate 18. Cover 26 substantially surrounds base 12 and flanged edges 24 of seal plate 18. Cover 26 fills a void between seal plate 18 and base 12, while also substantially covering flanged edges 24. Cover 26 thereby secures seal plate 18 to base 12 and blade 16 in base 12 without the use of adhesives, glue, bonding materials or fasteners that may add additional height or mass to bottom portion 20.

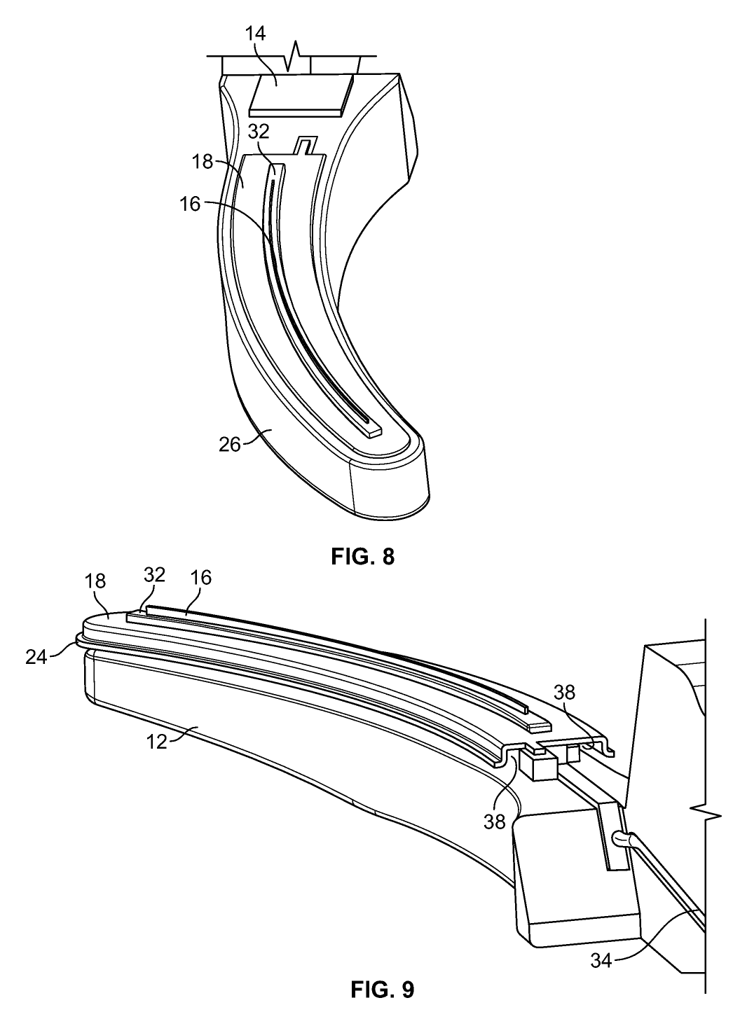

FIGS. 8 and 9 illustrate additional views of bottom portion 20 of the surgical jaw assembly. A space 38 is present between base 12 and seal plate 18 (FIG. 9) to provide an area for cover 26 to occupy when molded to bottom portion 20 (FIG. 8). Seal plate 18 may be electrically common with base 12 and may be plated with a material, e.g. gold. Alternatively, seal plate may be electrically isolated from base 12. Cover 26 may be approximately 0.030 inches thick, however, any suitable thickness may be used.

Surgical jaw assembly 10 may be manufactured or assembled by an exemplary method including the steps of providing a top portion 40 and a bottom portion 20, each having a base. An insert is disposed in the base and seal plate 18 is disposed substantially over the insert onto the base. The insert is exposed through an aperture in the seal plate. It is understood that the insert may protrude through the aperture, partially or completely. A cover is then molded substantially and at least partially over the base and seal plate. The molding of the cover onto the base and the seal plate secures the insert and seal plate to the base without the use of adhesives, glue, bonding materials, or fasteners. The molding process of the cover on the base may include an overmolding process or injection molding process, as well as any other suitable molding process.

Additionally, a blade 16 is disposed in a base and a seal plate 18 is disposed substantially over the blade onto the base. The blade protrudes through an aperture in the seal plate. A cover is then molded substantially and at least partially over the base and the seal plate. The molding of the cover onto the base and the seal plate secures the blade and seal plate to the base without the use of adhesives, glue, bonding material or fasteners. The blade may be molded with blade overmold before being disposed in the base. The blade overmold substantially fits into the aperture of the seal plate. The molding process of the cover on the base may include an overmolding process or injection molding process, as well as any other suitable molding process.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A surgical jaw assembly comprising:
a bottom portion comprising:
a first base;
a blade disposed in the first base;
a first seal plate disposed on the first base, the first seal plate having an aperture and a flanged edge; and
a first cover at least partially covering the first base;
a top portion comprising:
a second base;
an insert disposed in the second base;
a second seal plate disposed on the second base, the second seal plate having an aperture and a flanged edge; and
a second cover at least partially covering the second base;
wherein the first seal plate is in contact with the first base and the second seal plate is in contact with the second base, wherein the first cover and second cover covers at least a portion of the first base and at least a portion of the second base, wherein the first cover secures the blade and first seal plate to the first base by engaging the flanged edge of the seal plate, and wherein the second cover secures the insert and second seal plate to the second base by engaging the flanged edge of the seal plate.

2. The surgical jaw assembly of claim 1, wherein the first cover and second cover are plastic.

3. The surgical jaw assembly of claim 2, wherein the blade is an electrically charged blade.

4. The surgical jaw assembly of claim 1, wherein the first seal plate and first base are electrically common and where the second seal plate and second base are electrically common.

5. The surgical jaw assembly of claim 1, wherein the blade is stationary relative to the base.

6. The surgical jaw assembly of claim 1, wherein the blade is electrically charged.

7. The surgical jaw assembly of claim 1, wherein the first base further comprises a stop surface.

8. The surgical jaw assembly of claim 7, wherein the stop surface is unitary with the first cover.

9. The surgical jaw assembly of claim 7, wherein the stop surface is unitary with the first base.

10. The surgical jaw assembly of claim 1, wherein the second base further comprises a stop surface.

11. A method of manufacturing a surgical jaw assembly comprising the steps of:
providing a bottom portion having a first base, an blade, a first seal plate and a first cover, the blade being disposed in the first base and the first seal plate being disposed on the first base such that the first seal plate contacts the first base;
providing a top portion having a second base, an insert, a second seal plate and a second cover, the insert being disposed in the second base and the second seal plate being disposed on the second base such that the second seal plate contacts the second base;
overmolding a first cover at least partially surrounding the first base, the first cover securing the blade and first seal plate to the first base such that the first cover engages the first seal plate; and
overmolding a second cover at least partially surrounding the second base, the second cover securing the insert and second seal plate to the second base such that the second cover engages the second seal plate.

12. The method of claim 11, wherein at least one of the overmolding steps includes overmolding a plastic cover.

13. The method of claim 12, wherein at least one of the overmolding steps includes an injection molding process.

14. The method of claim 11, wherein the step of providing a bottom portion further comprises providing an electrically charged blade.

15. A method of manufacturing a surgical jaw assembly comprising the steps of:
providing at least one base;
aligning a seal plate having a flanged edge atop the at least one base, such that the seal plate is in contact with the at least one base; and
molding a cover at least partially surrounding the at least one base, the cover securing seal plate to the at least one base by engaging with the flanged edge of the seal plate.

16. The method of claim 15 comprising the step of disposing an electrically charged blade in the base before the step of aligning the seal plate on the at least one base.

17. The method of claim 15 comprising the step of disposing an insert in the base before the step of aligning the seal plate on the at least one base.

18. The method of claim 15, wherein the step of providing at least one base further comprises providing two bases such that the two bases are hingedly attached to one another.

19. The method of claim 15, wherein the molding step includes an overmolding process.

20. The method of claim 19, wherein the molding step includes an injection molding process.
